(19) United States
(12) Patent Application Publication
(10) Pub. No.: US 2026/0355494 A1
(43) Pub. Date: Nov. 12, 2026

(54) VALVE CONTROL DEVICE AND FLUID PATH CONTROL METHOD

(71) Applicant: NAVIGANTIS, INC., Miami, FL (US)

(72) Inventors: Ji Hyun PARK, Ansan-si (KR); Chul Hyun JOO, Bucheon-si (KR); Nam Ho SHIN, Incheon (KR)

(73) Assignee: NAVIGANTIS, INC., Miami, FL (US)

(21) Appl. No.: 19/663,730

(22) Filed: Apr. 30, 2026

(30) Foreign Application Priority Data
Apr. 30, 2025 (KR) 10-2025-0057698
Apr. 28, 2026 (KR) 10-2026-0076632

(51) Int. Cl. A61M 39/22; A61M 5/00; A61M 5/14; A61M 5/145; A61M 5/168; A61M 5/172

(52) U.S. Cl. CPC A61M 39/223; A61M 5/007; A61M 5/1408; A61M 5/1452; A61M 5/16813; A61M 5/172; A61M 2039/226; A61M 2205/0272; A61M 2205/103; A61M 2205/332; A61M 2205/3327; A61M 2205/50

(57) ABSTRACT

A valve control device according to the disclosed embodiment includes an actuator configured to provide a driving force for rotating a handle of a three-way stopcock valve, a valve fixing part configured to contact an outer surface of a lumen housing of the stopcock valve to fix the lumen housing, and a power transmission coupler configured to transmit a rotational force generated in an output shaft of the actuator to the handle.

### CROSS-REFERENCE TO RELATED APPLICATION

This application is based upon and claims the benefit of priority from Korean Patent Application No. 10-2025-0057698, filed on Apr.30, 2025, and Korean Patent Application No. 10-2026-0076632, filed on Apr. 28, 2026, which claims priority to the aforementioned provisional application, the entire contents of which are incorporated herein by reference.

### TECHNICAL FIELD

The present disclosure relates to a three-way stopcock valve control device used in a vascular interventional procedure, and a fluid path control method using the same.

### BACKGROUND

The vascular interventional procedure is a medical procedure in which a catheter is inserted into a patient's blood vessel to perform a diagnosis or a treatment, and includes various types of medical procedures such as angiography, embolization, and thrombectomy. In such a medical procedure, a plurality of fluids, such as a contrast agent, a saline solution, and an embolic material, are injected into the patient's blood vessel through the catheter, and a three-way stopcock valve is used to switch the fluid path.

In a vascular interventional procedure of related art, the switching of a handle of a stopcock valve has been performed manually by an operator or an assistant. During the procedure, the valve handle needs to be switched frequently while repeating steps such as contrast agent supply, saline solution flushing, and air extraction. This increases the workload on the operator, poses a risk of infection due to manual operation, and limits the accuracy and reproducibility of the valve switching.

In particular, as the introduction of a robotic system has expanded, the advancement and rotation of the catheter can be remotely controlled by the robot. However, the valve operation for fluid path switching is still performed manually, which is a factor hindering the complete automation of the procedure.

Therefore, a need has existed for a valve control device that can be attached to a commercially available stopcock valve without structural changes and can automatically switch a valve handle while maintaining a sterile environment.

### SUMMARY

Various embodiments of the present disclosure solve the problems inherent in the related art described above.

Various embodiments of the present disclosure provide a valve control device that can be attached to and detached from a commercially available three-way stopcock valve without structural changes and can automatically rotate a valve handle while maintaining a sterile environment.

Further, various embodiments of the present disclosure provide a fluid management system capable of automatic switching of fluid paths in procedures such as angiography, embolization, and thrombectomy by sequence-controlling the handle switching of a plurality of stopcock valves connected in series.

A valve control device according to one embodiment for rotating a handle of a three-way stopcock valve includes: an actuator configured to provide a driving force for rotating the handle; a valve fixing part configured to contact an outer surface of a lumen housing of the stopcock valve to fix the lumen housing; and a power transmission coupler configured to transmit the driving force of the actuator to the handle.

In one embodiment, the device may be integrated into a robot system, and may further include: a controller configured to receive a fluid flow switching command from a robot controller of the robot system and to rotate the handle to a target angle by driving the actuator in response to the fluid flow switching command.

In one embodiment, the device may further include: a sensor configured to detect a rotation state of the handle, wherein the controller may be configured to determine an initial position of the handle based on the rotation state detected by the sensor, calculate an amount of rotation from the initial position to a target position, and input a driving signal corresponding to the amount of rotation to the actuator.

In one embodiment, the power transmission coupler may include a disc coupled to an output shaft of the actuator and at least one driving pin arranged at an eccentric position radially spaced apart by a predetermined distance from a center of rotation of the output shaft on an upper surface of the disc, and configured to apply a driving force by contacting the handle.

In one embodiment, the sensor may be a torque sensor configured to detect a load torque when the handle is driven. The controller may be configured to monitor a driving torque from the torque sensor while rotating the driving pin, and to calculate the initial position of the handle by detecting an angle at which the driving torque exceeds a predetermined threshold value.

In one embodiment, the driving pin may be one, the handle may have a T-shaped branch structure, and the driving pin may be arranged on the disc so as to be located within a sector where the angular spacing between two adjacent branches of the handle is 180 degrees.

In one embodiment, the driving pin may include a first driving pin and a second driving pin arranged on the disc at an angular spacing of 90 degrees from each other, and the controller may be configured to calculate the initial position of the handle based on whether a magnitude of the torque detected when the disc is rotated clockwise and counterclockwise is a first torque generated by a single contact between either the first driving pin or the second driving pin and the handle, or a second torque generated by a dual contact between both the first driving pin and the second driving pin and the handle.

In one embodiment, the device may further include: a sterile barrier configured to form a physical boundary between a sterile region and a non-sterile region, wherein the valve fixing part and the power transmission coupler may be arranged in the sterile region, and the actuator may be arranged in the non-sterile region.

In one embodiment, the power transmission coupler may include a magnetic coupler, the magnetic coupler may include a motor-side element including a driving magnet coupled to the output shaft of the actuator and arranged in the non-sterile region, and a driven magnet arranged in the sterile region opposite the driving magnet and configured to transmit a torque to the handle. The sterile barrier may be interposed between the driving magnet and the driven magnet so that a torque can be transmitted by a magnetic force without physical contact.

In one embodiment, at least one of the valve fixing part, the power transmission coupler, or the actuator may be provided by being integrated into a disposable sterile cassette.

In one embodiment, the power transmission coupler may include a first pulley mounted on an output shaft of a motor, a second pulley connected to a rotary shaft of the handle, and a belt stretched between the first pulley and the second pulley to transmit rotational motion.

In one embodiment, the power transmission coupler may include a worm gear connected to an output shaft of a motor, and a worm wheel gear driven by meshing with the worm gear and configured to transmit a rotational force to the handle.

In one embodiment, the valve fixing part may be coupled to a main body rotatably around a hinge shaft. A plurality of fixing pins may be formed on an inner surface of the valve fixing part to contact the outer surface of the lumen housing and to restrain the lumen housing in rotational and lateral directions. When the valve fixing part is closed by being rotated toward the main body, the lumen housing may be fitted and fixed between the plurality of fixing pins, and the handle may engage with the power transmission coupler.

A valve control device according to another embodiment includes a plurality of valve control devices for rotating handles of a plurality of three-way stopcock valves, respectively. Each of the plurality of valve control devices may include: an actuator configured to provide a driving force for rotating a corresponding handle; a valve fixing part configured to contact an outer surface of a lumen housing of a corresponding stopcock valve to fix the lumen housing; and a power transmission coupler configured to transmit the driving force of the actuator to the corresponding handle.

In one embodiment, the plurality of three-way stopcock valves may include a first stopcock valve and a second stopcock valve connected in series with the first stopcock valve. The plurality of valve control devices may include a first valve control device configured to rotate a handle of the first stopcock valve, and a second valve control device configured to rotate a handle of the second stopcock valve. Each of the first stopcock valve and the second stopcock valve may include a second port on a straight flow path located upstream in a flow direction of a fluid flowing toward a first catheter, a third port located downstream in the flow direction and arranged opposite the second port on the straight flow path, and a first port branched in a direction intersecting the straight flow path between the second port and the third port. A manual syringe line with a syringe connected thereto may be connected to the second port of the first stopcock valve, an automatic contrast agent injector line with a power injector connected thereto may be connected to the first port of the first stopcock valve, and the third port of the first stopcock valve may be fluidly connected to the second port of the second stopcock valve. A saline solution line may be connected to the first port of the second stopcock valve, and an injection line fluidly connected to the first catheter may be connected to the third port of the second stopcock valve. The valve control device may further include a controller configured to perform a first control sequence that sequentially switches a fluid path communicating with the injection line by transmitting a control command to the first valve control device and the second valve control device and rotating each handle of the first stopcock valve and the second stopcock valve.

In one embodiment, the first control sequence may include a first step of rotating the first stopcock valve into a posture in which the manual syringe line communicates with the second port of the second stopcock valve, and rotating the second stopcock valve into a posture in which the second port communicates with the injection line, to thereby form a fluid path extending from the syringe to the injection line so that air within the fluid path can be extracted.

In one embodiment, the first control sequence may further include a second step of, after the first step, rotating the first stopcock valve to block communication between the manual syringe line and the automatic contrast agent injector line and the second port of the second stopcock valve, and rotating the second stopcock valve to bring the saline solution line into communication with the injection line, so that a saline solution can be supplied from the saline solution line toward the first catheter.

In one embodiment, the first control sequence may further include a third step of, after the second step, rotating the second stopcock valve to block communication between the saline solution line and the injection line and to bring the second port of the second stopcock valve into communication with the injection line, and rotating the first stopcock valve to form a posture in which the manual syringe line communicates with the injection line via the second ports of the first stopcock valve and the second stopcock valve, so that a contrast agent can be supplied from the syringe toward the first catheter.

In one embodiment, the first control sequence may further include a fourth step of, after the third step, rotating the first stopcock valve while maintaining the posture of the second stopcock valve to block the manual syringe line and to bring the automatic contrast agent injector line into communication with the second port of the second stopcock valve, so that the contrast agent can be injected from the power injector toward the first catheter through the injection line.

In one embodiment, the first control sequence may further include a fifth step of, after the fourth step, rotating the first stopcock valve to block both the automatic contrast agent injector line and the manual syringe line, and rotating the second stopcock valve to allow the saline solution line to communicate with the injection line again.

In one embodiment, the first control sequence may repeatedly perform a cycle including the second step, the third step or the fourth step, and the fifth step.

In one embodiment, the plurality of valve control devices may further include a third valve control device configured to rotate a handle of a third stopcock valve arranged on the side of a second catheter. The third stopcock valve may include a second port on a straight flow path located upstream in a flow direction of a fluid flowing toward the second catheter, a third port located downstream in the flow direction of the fluid flowing toward the second catheter and arranged opposite the second port of the third stopcock valve on the straight flow path, and a first port branched in a direction intersecting the straight flow path between the second port and the third port of the third stopcock valve. A manual syringe line with a syringe connected thereto may be connected to the second port of the third stopcock valve, a saline solution line may be connected to the first port of the third stopcock valve, and an injection line fluidly connected to the second catheter via a rotary hemostatic valve may be connected to the third port of the third stopcock valve. The controller may be configured to, after the first control sequence, further perform a second control sequence that sequentially switches a fluid path communicating with the second catheter by transmitting a control command to the third valve control device and rotating the handle of the third stopcock valve.

In one embodiment, the second control sequence may include a sixth step of rotating the third stopcock valve to form a posture in which the second port and the third port are in communication with each other while blocking the first port, so that air can be extracted from a fluid path extending from the syringe to the second catheter through the rotary hemostatic valve.

In one embodiment, the second control sequence may further include a seventh step of, after the sixth step, rotating the third stopcock valve to block the second port and to bring the first port and the third port into communication with each other, so that a saline solution can be supplied from the saline solution line into the second catheter via the rotary hemostatic valve.

In one embodiment, the second control sequence may further include an eighth step of, after the seventh step, rotating the third stopcock valve to block the first port and to bring the second port and the third port into communication with each other, so that a contrast agent or an embolic material can be injected from the syringe into the second catheter via the rotary hemostatic valve.

In one embodiment, the second control sequence may repeatedly perform a cycle including the seventh step and the eighth step.

A fluid path control method according to another embodiment is a fluid path control method for switching a fluid path communicating with an injection line connected to a first catheter by rotating each handle of a first three-way stopcock valve and a second three-way stopcock valve connected in series by a valve control device. The fluid path control method may include a first control sequence in which a controller automatically switches the handle of the first stopcock valve and the handle of the second stopcock valve in a predetermined order.

In one embodiment, the method may further include a second control sequence that rotates a handle of a third stopcock valve arranged on the side of a second catheter by a third valve control device.

### BRIEF DESCRIPTION OF DRAWINGS

The accompanying drawings, which are incorporated in and constitute a part of the specification, illustrate embodiments of the present disclosure, and together with the general description given above and the detailed description of the embodiments given below, serve to explain the principles of the present disclosure.

FIG. 1 illustrates the overall configuration of a valve control device according to one embodiment.

FIG. 2 illustrates an application example in which the valve control device according to one embodiment is integrated into a sterile cassette of a robot system.

FIG. 3 illustrates an assembled structure of an actuator, an actuator rotation axis, and a driving pin according to one embodiment.

FIG. 4 illustrates a method for controlling a posture of a valve handle using a driving pin according to one embodiment.

FIG. 5 illustrates a principle of detecting an initial position of the valve handle based on a change in driving torque according to one embodiment.

FIG. 6 illustrates a method for determining the posture of the valve handle using a plurality of driving pins according to one embodiment.

FIG. 7 illustrates a pulley-type valve control device according to one embodiment.

FIG. 8 illustrates a worm gear-type valve control device according to another embodiment.

FIG. 9 illustrates a structure of a power transmission coupler according to one embodiment.

FIG. 10 illustrates a structure of a valve fixing part according to one embodiment.

FIG. 11 illustrates an automatic chuck type valve fixing part according to another embodiment.

FIG. 12 illustrates a fluid path configuration including a plurality of stopcock valves connected in series according to one embodiment.

FIG. 13 illustrates an air extraction step of a valve sequence control method during angiography according to one embodiment.

FIG. 14 illustrates a saline solution connection step of the valve sequence control method during angiography according to one embodiment.

FIG. 15 illustrates a test contrast agent supply step by a syringe in the valve sequence control method during angiography according to one embodiment.

FIG. 16 illustrates a contrast agent supply step by a power injector in the valve sequence control method during angiography according to one embodiment.

FIG. 17 illustrates a saline solution reconnection step in the valve sequence control method during angiography according to one embodiment.

FIG. 18 illustrates a step of extracting air from a microcatheter-side line in the valve sequence control method during embolization and thrombectomy according to one embodiment.

FIG. 19 illustrates a step of connecting a saline solution to the microcatheter-side line in the valve sequence control method during embolization and thrombectomy according to one embodiment.

FIG. 20 illustrates a step of injecting a contrast agent or an embolic material through the microcatheter-side line in the valve sequence control method during embolization and thrombectomy according to one embodiment.

FIG. 21 is a flowchart showing an overall flow of a fluid path control method according to one embodiment.

### DETAILED DESCRIPTION

Embodiments of the present disclosure are illustrated for the purpose of explaining the technical idea of the present disclosure. The scope of the rights according to the present disclosure is not limited to the embodiments presented below or the detailed descriptions of such embodiments.

All the technical terms and scientific terms in the present disclosure include meanings or definitions that are commonly understood by those of ordinary skill in the art unless otherwise defined. All terms in the present disclosure are selected for the purpose of describing the present disclosure more clearly, and are not selected to limit the scope of the present disclosure.

As used in the present disclosure, expressions such as "comprising," "including," "having," and the like are to be understood as open-ended terms having the possibility of encompassing other embodiments, unless otherwise mentioned in the phrase or sentence containing such expressions.

Singular expressions that are described in the present disclosure may encompass plural expressions unless otherwise stated, which also apply to the singular expressions recited in the claims.

The expressions, such as "first," "second," etc., which are shown in various embodiments of the present disclosure, are used to separate a plurality of elements from each other, and are not intended to limit the order or importance of the corresponding elements.

In the present disclosure, the expression "based on" will be used to describe one or more factors that affect an act or operation of a decision or determination that is described in the phrase that contains the corresponding expression, and does not exclude additional factors that affect the act or operation of the decision or determination.

In the present disclosure, the description that one element is "connected" or "coupled" to another element should be understood to indicate that one element may be directly connected or coupled to another element, and should be further understood that a new element may be interposed between one element and another element.

Hereinafter, the embodiments of the present disclosure will be described in detail with reference to the accompanying drawings. The same reference numeral will be used for the identical or corresponding element throughout the drawings. Furthermore, in the following descriptions of embodiments, the descriptions of identical or corresponding elements may be omitted. However, even if the descriptions of elements are omitted, it is not intended that such elements are not included in a certain embodiment.

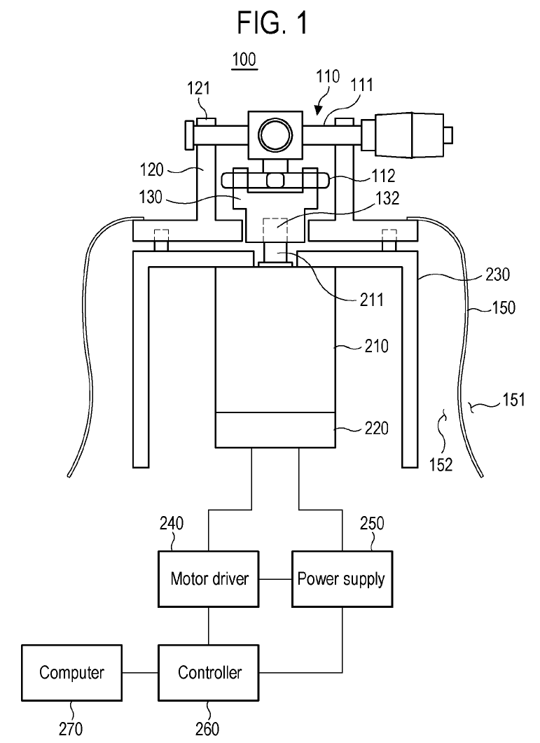

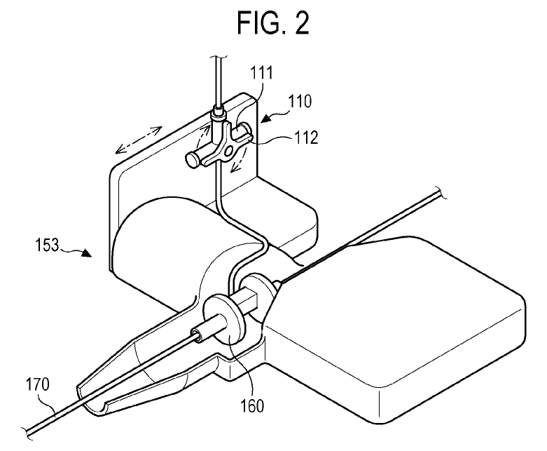

FIG. 1 illustrates an overall configuration of a valve control device according to one embodiment. FIG. 2 illustrates an application example in which the valve control device according to one embodiment is integrated into a sterile cassette of a robot system.

Referring to FIG. 1, the valve control device 100 includes sterile-side components arranged in a sterile region 151, non-sterile-side components arranged in a non-sterile region 152, and a sterile barrier 150 interposed between the two regions. In a vascular interventional procedure, the valve control device 100 performs a function of automatically switching a fluid flow path while maintaining the boundary between a sterile surgical environment and an electromechanical drive system. Specifically, the valve control device 100 can switch the fluid flow path without the operator’s manual operation by automatically rotating the handle 112 of a stopcock valve 110.

The sterile-side components include a three-way stopcock valve 110, a valve fixing part 120, and a power transmission coupler 130. The three-way stopcock valve 110 includes a lumen housing 111 having a T-shaped port structure, and a handle 112 rotatably installed with respect to the lumen housing 111, wherein a fluid communication path between ports is selectively switched depending on the rotational position of the handle 112. The three-way stopcock valve 110 may be a commercially available product commonly used in vascular interventional procedures, and the valve control device 100 of the present disclosure is designed to be attached to and detached from such a commercially available valve without structural changes. In the present disclosure, the term "stopcock valve" means a three-way stopcock valve.

The valve fixing part 120 is a mechanism for fixing the lumen housing 111 of the stopcock valve 110 at a predetermined position and in a predetermined posture with respect to an actuator housing 230. The valve fixing part 120 may include various types of fixing elements 121 that contact the outer surface of the lumen housing 111, and may be implemented, for example, as a clamp, snap-fit, slot insertion structure, etc.

The power transmission coupler 130 is configured to transmit a rotational force generated from an actuator 210 in the non-sterile region 152 to the handle 112 in the sterile region 151. The power transmission coupler 130 may receive a torque by being directly connected to the output shaft 211 of the actuator 210, or may receive a torque through a motor-side element 132 coupled to the output shaft 211 and transmit the torque to the handle 112. The motor-side element 132 is an intermediary element arranged on the side of the non-sterile region 152 to transmit a torque from the output shaft 211 to the power transmission coupler 130. In a non-contact type embodiment described below, the motor-side element 132 may be implemented in a form including a driving magnet, and in a contact type embodiment described below, the motor-side element 132 may be implemented as a mechanical coupling element (e.g., a coupling hub).

In one embodiment, the power transmission coupler 130 may be implemented as a non-contact coupler including a magnetic coupler. In this case, the motor-side element 132 including a driving magnet coupled to the output shaft 211 is arranged on the non-sterile region 152 side, and the power transmission coupler 130 including a driven magnet positioned opposite to the driving magnet is arranged on the sterile region 151 side. The sterile barrier 150 is interposed between the driving magnet and the driven magnet so that a torque can be transmitted by a magnetic force without physical contact. The driven magnet transmits a torque to the handle 112 in the sterile region 151. This non-contact system simplifies the sealing structure of the sterile barrier 150 and reduces the wear of barrier members.

In another embodiment, the output shaft 211 may be implemented as a contact type that is directly connected to the power transmission coupler 130. In this case, the output shaft 211 is configured as a sealed penetration shaft that penetrates the sterile barrier 150, and sterilization may be maintained by a sealing member such as an O-ring or a lip seal. In addition, when the rotational axes of the output shaft 211 and the handle 112 are non-coaxial, power transmission means such as a belt and pulley, a gear train or a flexible shaft, etc. may be additionally provided between the motor-side element 132 and the power transmission coupler 130, or between the power transmission coupler 130 and the handle 112.

The sterile barrier 150 forms a physical boundary between the sterile region 151 and the non-sterile region 152, thereby preventing microorganisms or foreign substances from entering the sterile region 151 during the procedure. The sterile barrier 150 may be implemented in the form of a drape-type cover or a rigid cassette. In particular, when the sterile barrier 150 is implemented in the form of a cassette, it can be provided as a disposable sterile cassette 153. That is, the disposable sterile cassette 153 is one implementation of the sterile barrier 150, which accommodates the valve fixing part 120, the power transmission coupler 130, and the stopcock valve 110 in a pre-assembled state.

The non-sterile-side components include an actuator 210, a sensor 220, an actuator housing 230, a motor driver 240, a power supply 250, and a controller 260. The actuator 210 is a driving source that generates a driving force to rotate the handle 112, and may be implemented as a stepping motor, a DC brushless motor, a servo motor, etc. The sensor 220 is a sensing means that detects the rotation state of the handle 112. In the present disclosure, the term "rotation state" is a concept that encompasses the rotational angle, the rotational position, and the posture information of the handle 112 that can be derived from them. The controller 260 determines the initial position of the handle 112 based on the rotation state detected by the sensor 220, calculates the amount of rotation from the initial position to a target position, and inputs a driving signal corresponding to the amount of rotation to the actuator 210. The motor driver 240 is a device that converts a signal generated by the controller 260 into an actual torque to move the actuator 210 as desired. The controller 260 can detect changes in current generated in the motor driver 240 when an external load is applied to the actuator 210, thereby estimating the presence or magnitude of the external force. Driving commands for the actuator 210 from the controller 260 can be output in a form that passes through the motor driver 240 or in a form in which the controller 260 directly controls the actuator 210. Additionally, the controller 260 may receive control commands from a computer 270 which is an external system. The computer 270 may be a device including control software running on a robot control computer, a surgical workstation, or a general-purpose computer, and transmits fluid flow switching commands input by the operator via a GUI or a physical input means to the controller 260.

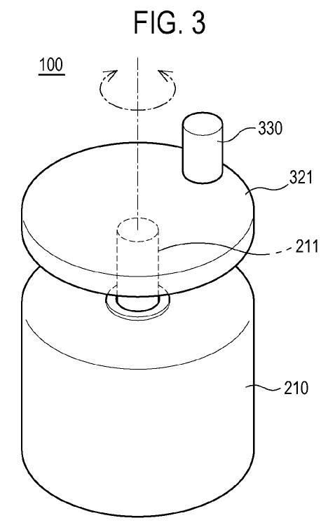

Meanwhile, the valve fixing part 120 and the power transmission coupler 130 may be configured to mechanically align the handle 112 at a predetermined reference position when the stopcock valve 110 is mounted. By doing so, the controller 260 can control the rotation of the handle 112 from a consistent reference position without a separate initial position detection process. In another embodiment, an initial position detection process using the sensor 220 may be performed instead of mechanical pre-alignment, which will be described in detail later with reference to FIGS. 3, 4 and 5.

At least the part of the valve control device 100 that can come into contact with a patient may be made of a material suitable for low-temperature sterilization. Specifically, use of a material capable of maintaining shape stability at about 40 to 50 degrees C or lower, such as engineering plastics (e.g., PEEK and polycarbonate), stainless steel, medical silicone, etc., makes it possible to perform sterilization in a low-temperature plasma sterilizer.

Referring to FIGS. 1 and 2, the valve control device 100 may be operated independently as a standalone device or may be provided while being integrated into a robotic system for vascular interventional procedures.

When operated as a standalone device, the valve control device 100 may be fixed by being suspended from the rail or frame of an angiography table with a clamp or bracket, or may be placed on a separate stand.

In the present disclosure, the phrase "the valve control device 100 integrated into the robot system" means that at least some of the components of the valve control device 100 are mechanically mounted or built in the robot system. For example, the non-sterile-side components such as the actuator 210, the motor driver 240, and the controller 260 may be built in the robot body or robot flange, and the sterile-side components such as the valve fixing part 120 and the power transmission coupler 130 may be included in the disposable sterile cassette 153. The disposable sterile cassette 153 is supplied in a sterile packaged state before the procedure and may contain the stopcock valve 110, the tubing path, the valve fixing part 120, and the power transmission coupler 130 in a pre-assembled state, which makes it possible to reduce the procedure preparation time. In addition, the controller 260 may be provided in a state in which it can receive control commands by being connected to the robot controller of the robot system via a communication interface. In this way, by physically combining the valve control device 100 with the robot system and linking the valve control device 100 to the robot system through signals, the valve control device 100 can function as a component module of the robot system.

In this integrated configuration, the stopcock valve 110 may be arranged to move together with a rotary hemostatic valve 160. Fluid path alignment between the stopcock valve 110 and the rotary hemostatic valve 160 can be maintained even while the robot advances or rotates a first catheter 170. The first catheter 170 is a tubular medical device inserted into a patient's blood vessel and can be brought into fluid communication with the stopcock valve 110.

When the valve control device 100 is integrated into the robot system, the controller 260 may receive a fluid flow switching command from the robot controller. In the present disclosure, the term "fluid flow switching command" means a control signal that includes, directly or indirectly, information regarding the target rotational position of the handle 112 to switch the fluid path provided by the three-way stopcock valve 110 from a current state to another state. Specifically, the fluid flow switching command may be configured in the form of a numerical designation of a target angle of the handle 112, or in the form of a mode identifier that identifies fluid flow modes, e.g., a saline solution supply mode, a contrast agent supply mode, an air extraction mode, etc. In the case of the form of the mode identifier, the controller 260 may determine a corresponding target angle from a pre-stored lookup table based on the received mode identifier. When the controller 260 receives a fluid flow switching command, it calculates the amount of rotation between the current position of the handle 112 and the target angle based on the output signal of the sensor 220, and outputs a driving signal corresponding to the calculated amount of rotation to the actuator 210 to rotate the handle 112 to the target angle. Thus, when the operator selects a desired fluid flow mode on the robot control interface, the remote control of the first catheter 170 and the switching of the fluid path can be performed integrally.

The valve control device 100 may be arranged in a form in which a single actuator 210 corresponding to a single valve exists as an individual device, in a form in which a plurality of actuators 210 corresponding to a plurality of valves are integrated into a single module housing, or in a form in which a plurality of actuators 210 are mounted on a side surface of an angiography table.

When used alone without a robot system, the user can directly specify the valve position through an input part provided in the actuator housing 230 or a separate input device.

FIG. 3 illustrates an assembled structure of the actuator, the rotary shaft of the actuator, and the driving pin according to one embodiment.

Referring to FIG. 3, the valve control device 100 includes an actuator 210 configured to generate a rotational force, an output shaft 211 of the actuator 210, a disc 321 fixed to the upper portion of the output shaft 211, and at least one driving pin 330 fixed at an eccentric position on the upper surface of the disc 321. The combination of the disc 321 and the driving pin 330 can constitute an embodiment of the power transmission coupler 130 shown in FIG. 1. That is, the power transmission coupler 130 may be implemented in such a way that the rotational force generated from the output shaft 211 of the actuator 210 is transmitted to the handle 112 by using the driving pin 330. The output shaft 211 is coupled to the rotation center of the disc 321 to rotate the disc 321 and the driving pin 330 as a single unit.

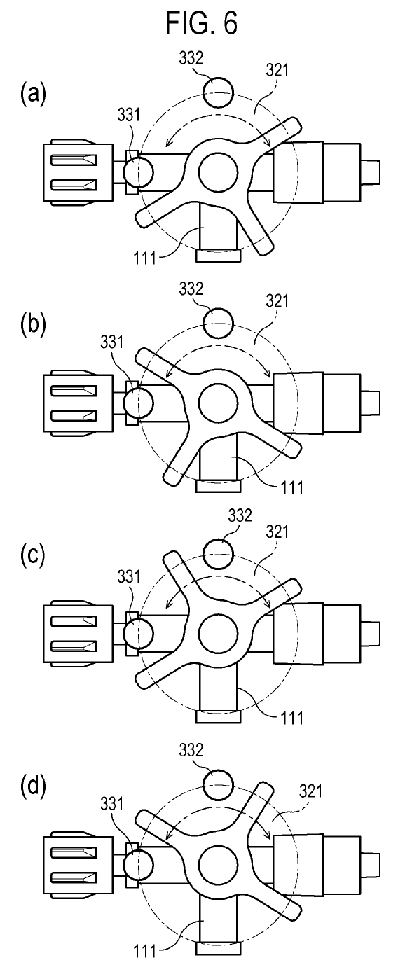

The driving pin 330 is a protruding member for applying a rotational force to the handle 112 by contacting the branch of the handle 112, and is arranged at a position radially spaced apart by a predetermined distance from the center of rotation of the output shaft 211. Due to this eccentric arrangement, when the output shaft 211 rotates, the driving pin 330 moves along a circular trajectory surrounding the center of rotation. Although FIG. 3 illustrates a case where there is one driving pin 330, two or more driving pins 330 may be arranged on the upper surface of the disc 321. An embodiment using a plurality of driving pins 330 will be described later with reference to FIG. 6. The shape of the driving pin 330 may be cylindrical or polygonal, or may have a rounded tip. The contact surface of the driving pin 330 may be smoothed, formed of an elastic member, or coated with an elastic member to prevent damage otherwise caused by contact with a branch of the handle 112.

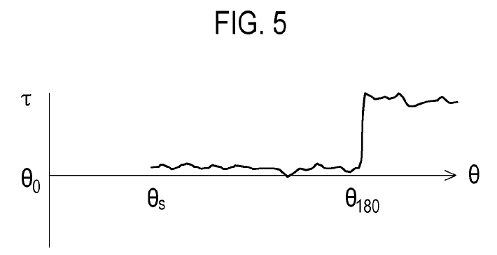

The sensor 220 may be a torque sensor capable of detecting a load torque when driving the handle. The torque sensor may be mounted directly on the output shaft 211 or may be built inside the actuator 210. Alternatively or additionally, the load torque may be measured by measuring the magnitude of the current output from the motor driver. The change in load torque detected by the torque sensor is utilized for detecting an initial position of the handle, which will be described later with reference to FIG. 5, and is utilized for determining a posture of the handle, which will be described later with reference to FIG. 6.

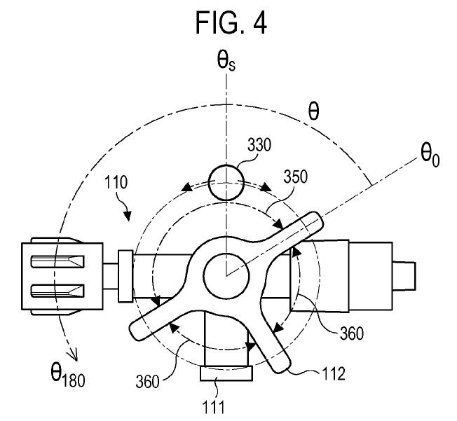

FIG. 4 illustrates a method for controlling a posture of the valve handle using the driving pin according to one embodiment. FIG. 4 is a plan view showing the driving pin 330 acting on the handle 112 by the rotation of the output shaft 211 of the actuator shown in FIG. 3.

Referring to FIG. 4, when the output shaft 211 rotates, the driving pin 330 moves along a circular trajectory and contacts the T-shaped branch portion of the handle 112 to apply a rotational force to the handle 112. This system may be applied to various handle shapes without a separate coupling structure that matches the shape of the handle.

The detection of an initial position of the handle 112 can be performed by at least one of the following methods. The first method is a mechanical indexing method in which the valve is configured to be coupled in only one direction by the shape of the valve fixing part 120, and the handle 112 is forcibly aligned at a reference angle by the power transmission coupler 130, so that the initial position can be determined without a separate sensor. The second method is a torque-based haptic mapping method in which the driving torque of the actuator 210 is continuously monitored while the driving pin 330 is rotated at a low speed, and the angle at which the torque exceeds a predetermined threshold value is detected to estimate a branch position of the handle 112. The torque-based haptic mapping method includes a case where the driving pin 330 is composed of a single driving pin 330 and a case where the driving pin 330 is composed of a plurality of driving pins 331 and 332. Details of an embodiment using a single driving pin 330 will be described later with reference to FIG. 5, and details of an embodiment using a plurality of driving pins 331 and 332 will be described later with reference to FIG. 6. The third method is an optical detection method in which a camera captures the handle 112 and calculates a handle angle by an image processing algorithm (e.g., edge detection and template matching). The fourth method is a gravity-based detection method in which an inclination angle of the handle 112 with respect to a gravity acceleration vector is calculated from an output of an IMU mounted on the handle 112 or on a cap-shaped plate attached to the handle 112.

When the initial position of the handle 112 is determined, the controller 260 calculates an amount of rotation from the initial position to the target position according to a command entered by the operator through the GUI or a preset workflow sequence, and inputs a driving signal corresponding to the amount of rotation to the actuator 210 to rotate the handle 112 to the target position.

The valve control device 100 has a reversible structure that allows it to be attached to and detached from the stopcock valve 110 without structural changes. Therefore, after detachment of the valve control device 100, the valve is restored to its original state and can be manually operated. In addition, a manual override mode can be provided even when the valve control device 100 is installed. The manual override mode is implemented by cutting off a current, releasing an electronic clutch, or releasing a software torque limit. In the manual override mode, the operator can directly rotate the handle 112. Therefore, the safety of the procedure is maintained even under an emergency situation such as a malfunction of an automatic control system or a communication failure.

FIG. 5 illustrates a principle of detecting an initial position of the valve handle based on a change in driving torque according to one embodiment.

The handle 112 of the three-way stopcock valve 110 has a T-shaped branch structure consisting of three branches. Two of the three branches opposing each other are arranged at an angular spacing of 180 degrees, and the remaining branch is arranged at an angular spacing of 90 degrees to each of the two opposing branches. Due to this T-shaped geometry, three sectors 350 and 360 are formed in a circumferential direction of the handle 112 with angular spacings between the branches of 90 degrees, 90 degrees, and 180 degrees.

When there is only one driving pin 330, the driving pin 330 is assembled so that it is positioned within the sector 350 where the spacing between the branches in the circular trajectory is 180 degrees. By positioning the driving pin 330 within the 180-degree sector 350, the branch direction of the handle 112 can be clearly determined from an angle at which a torque surge occurs, which makes it possible to reliably grasp the accurate rotation state of the handle 112 and the open/closed state of the valve.

The lumen housing 111 is coupled only in a specific direction by the valve fixing part 120, and an assembly position of the disc 321 and the driving pin 330 is predetermined with respect to the valve fixing part 120. Therefore, an initial angular position θ_{S} of the driving pin 330 with respect to the lumen housing 111 at the time at which the setup is completed may be given as a predetermined value. That is, the angle θ_{S} is a value representing the initial position of the driving pin 330 and serves as a reference coordinate in the subsequent calculation of the handle position.

Referring to FIG. 5, the horizontal axis represents the rotation angle θ of the driving pin 330, and the vertical axis represents the driving torque τ of the actuator 210. The controller 260 can continuously monitor a driving torque signal received from the torque sensor while rotating the driving pin 330 clockwise and counterclockwise, respectively, from the initial position θ_{S}. In the section where the driving pin 330 does not contact the branch of the handle 112, the driving torque is maintained at a base level due to bearing friction or the like. However, at the moment the driving pin 330 contacts the branch of the handle 112, the driving torque rises rapidly due to the rotational resistance of the handle 112.

In one embodiment, an angle at which the driving torque exceeds a predetermined threshold value during clockwise rotation is detected as angle θ₀, and an angle at which the driving torque exceeds a predetermined threshold value during counterclockwise rotation is detected as angle θ₁₈₀. The angles θ₀ and θ₁₈₀ correspond to angular positions at the time when the driving pin 330 contacts two different branches of the handle 112.

The controller 260 can detect what posture the handle 112 is currently in, i.e., in which direction the T-shaped branch is facing relative to the lumen housing 111, by calculating the angle difference between the detected angle θ₀ or the angle θ₁₈₀ and the initial position θ_{S}. Since the posture of the handle 112 directly corresponds to the fluid communication path in the three-way stopcock valve 110, i.e., the open/closed state of the valve, the current state of the valve can be determined by calculating this angle difference.

The torque detection can be performed by at least one of a function of measuring a motor current by a current sensor built into the actuator 210, a function of directly measuring a torque by a torque sensor mounted on the output shaft 211, or a function of monitoring a current of the motor driver 240.

FIG. 6 illustrates a method for determining a posture of the valve handle using a plurality of driving pins according to one embodiment.

Referring to FIG. 6, a first driving pin 331 and a second driving pin 332 are arranged at an angular spacing of 90 degrees on the disc 321 mounted on the output shaft 211. The first driving pin 331 and the second driving pin 332 are configured so that when the disc 321 is coupled to the lumen housing 111, the disc 321 can always be assembled in the same reference direction relative to the lumen housing 111 due to the coupling structure between the disc 321 and the lumen housing 111. Accordingly, at the time when the disc 321 is mounted on the lumen housing 111, the relative arrangement between the first driving pin 331 and the second driving pin 332 and the T-shaped branches of the handle 112 corresponds to one of the following four cases (a) to (d).

Case (a) is an arrangement in which the first driving pin 331 and the second driving pin 332 do not come into contact with any branch of the handle 112, and only one driving pin can come into contact with the branch first in any rotational direction of the disc 321. In this case, only one driving pin comes into contact with the branch of the handle 112 in both counterclockwise and clockwise rotations. Therefore, a relatively small torque (first torque) can be detected in both directions.

Case (b) is an arrangement in which when the disc 321 is rotated counterclockwise, the first driving pin 331 and the second driving pin 332 come into contact with two adjacent branches of the handle 112 almost simultaneously. In this case, during a counterclockwise rotation, the two driving pins push the branches of the handle 112 simultaneously, so that a large torque (second torque) can be detected, and during a clockwise rotation, only one driving pin comes into contact with the branch, so that a first torque can be detected.

Case (c) is an arrangement in which regardless of whether the disc 321 is rotated counterclockwise or clockwise, the two driving pins simultaneously contact the branches of the handle 112 in either direction. In this case, a second torque due to dual contact can be detected during rotations in both directions.

Case (d) is an arrangement in which when the disc 321 is rotated clockwise, the first driving pin 331 and the second driving pin 332 come into contact with two adjacent branches of the handle 112 almost simultaneously, which is a direction pattern opposite that of (b). In this case, a second torque can be detected during the clockwise rotation, and a first torque can be detected during the counterclockwise rotation.

Since the second torque during dual contact is significantly larger than the first torque during single contact, the currently available one of the four cases (a) to (d) of arrangement can be determined by comparing the torque magnitude patterns in both counterclockwise and clockwise directions. Based on the determination result of these two-way torque patterns, the controller 260 can calculate the initial position of the handle 112.

In summary, when only one of the first driving pin 331 and the second driving pin 332 contacts a branch of the handle 112, the first torque of a relatively small magnitude is detected, and when both the first driving pin 331 and the second driving pin 332 contact two adjacent branches of the handle 112 simultaneously, the second torque of a relatively large magnitude is detected. The controller 260 can determine whether the magnitude of the torque detected when the disc 321 is rotated clockwise and counterclockwise corresponds to the first torque or the second torque, can determine which of the four cases (a) to (d) of arrangement described above corresponds to the combination of the determination results in both directions, and can calculate the branch direction of the handle 112 corresponding to the determined case of arrangement as the initial position of the handle 112. By using the initial position calculation method based on such a combination of bidirectional torque patterns, constraints on the assembly direction of the disc 321 during setup can be eliminated, and the accuracy of determining the posture of the handle 112 can be improved.

Meanwhile, the number and arrangement angle of the driving pins 330 are not limited to the above-described embodiment. Variations may be adopted, such as arranging three driving pins 330 at 120-degree intervals, or arranging two driving pins 330 at angles other than 90 degrees (e.g., 60 degrees or 45 degrees). Depending on the number and arrangement angle of the driving pins 330, a resolution of the distinguishable handle posture and a degree of freedom of assembly may vary.

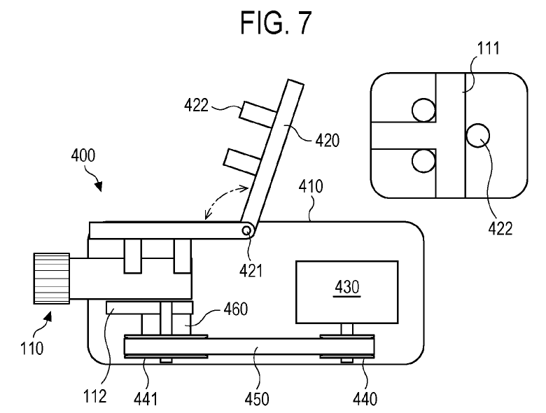

FIG. 7 illustrates a pulley-type valve control device according to one embodiment.

Referring to FIG. 7, the valve control device 400 may include a main body 410 and a valve fixing part 420. The main body 410 may be a structure on which power transmission components such as a motor 430, pulleys 440 and 441, a belt 450, and a power transmission coupler 460 are mounted. The valve fixing part 420 is a member that grips and fixes the lumen housing 111 of the stopcock valve 110, and may serve to fix the stopcock valve 110 to the main body 410.

A plurality of fixing pins 422 may be formed on the inner surface of the valve fixing part 420. When the stopcock valve 110 is seated on the valve fixing part 420, the lumen housing 111 is inserted between the plurality of fixing pins 422, so that the valve fixing part 420 and the lumen housing 111 can be coupled. By doing so, the stopcock valve 110 is stably constrained in the rotational and lateral directions, and the lumen housing 111 can be easily withdrawn from between the fixing pins 422 when the valve fixing part 420 is opened.

The valve fixing part 420 may be coupled to the main body 410 rotatably around a hinge shaft 421. When the valve fixing part 420 is rotated around the hinge shaft 421 away from the main body 410, it is opened to allow the stopcock valve 110 to be inserted into or withdrawn from the valve fixing part 420. After the stopcock valve 110 is seated in the valve fixing part 420, the valve fixing part 420 is closed by being rotated toward the main body 410. Then, the lumen housing 111 is inserted and fixed between the fixing pins 422, and the handle 112 of the stopcock valve 110 engages with the power transmission coupler 460 on the side of the main body 410, allowing the stopcock valve 110 to be fastened to the valve control device 400. Conversely, when the valve fixing part 420 is rotated in an opening direction, the insertion coupling between the lumen housing 111 and the fixing pins 422 and the engagement between the handle 112 and the power transmission coupler 460 are simultaneously released, allowing the stopcock valve 110 to be separated.

By adopting the rotary opening/closing structure using the hinge coupling as described above, the operator can simultaneously fix the position of the valve and connect the power through a single action of opening the valve fixing part 420, seating the stopcock valve 110, and then closing the valve fixing part 420. The closed state of the valve fixing part 420 can be maintained by at least one of a latch, snap-fit, magnetic attraction, or the frictional force of the hinge shaft 421.

A belt 450 may be stretched between a first pulley 440 mounted on the output shaft 431 of the motor 430 and a second pulley 441 connected to the rotary shaft of the handle 112 or connected to the handle 112 via the power transmission coupler 460 to transmit rotational motion. The pulley-belt system allows a position of the motor 430 to be spaced apart from the rotary shaft of the handle 112, which makes it possible to flexibly respond to spatial constraints around the valve. In addition, since the reduction ratio is determined by a diameter ratio of the first pulley 440 and the second pulley 441, a sufficient output torque can be obtained even with a small motor by forming the first pulley 440 on the motor side to have a small diameter and forming the second pulley 441 on the handle side to have a large diameter.

In another embodiment, the belt 450 may be replaced with a timing belt to enable slip-free precise rotation transmission. In yet another embodiment, spur gears, bevel gears, or flexible shafts may be used instead of the pulleys and the belt. Meanwhile, the coupling between the valve fixing part 420 and the main body 410 may be modified to sliding coupling, detachable clamp coupling, and the like, in addition to the hinge coupling.

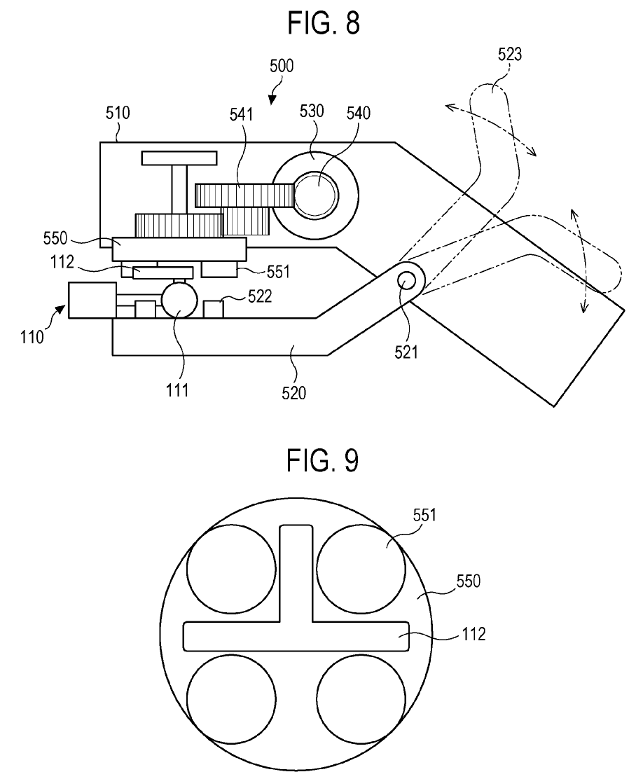

FIG. 8 illustrates a worm gear-type valve control device according to another embodiment.

Referring to FIG. 8, the valve control device 500 may include a main body 510 and a valve fixing part 520. The main body 510 may have a structure in which power transmission components such as a motor 530, a worm gear 540, a worm wheel gear 541, and a power transmission coupler 550 are mounted. The power transmission coupler 550 that transmits a rotational force by contacting an outer edge of the handle 112 may be arranged on a lower portion of the main body 510.

The valve fixing part 520 is a member that fixes the stopcock valve 110 by gripping the lower portion of the lumen housing 111 of the stopcock valve 110, and can be coupled to the main body 510 rotatably around a hinge shaft 521. A plurality of fixing pins 522 are arranged on the inner surface of the valve fixing part 520, so that the lumen housing 111 of the stopcock valve 110 can be stably fixed in the rotational and lateral directions by being fitted between the plurality of fixing pins 522. A handle 523 may be provided on the opposite side of the hinge shaft 521 for the operator to open and close the valve fixing part 520.

When the valve fixing part 520 is rotated away from the main body 510 around the hinge shaft 521, the insertion space for the stopcock valve 110 is opened, allowing the stopcock valve 110 to be inserted or withdrawn. After the stopcock valve 110 is seated on the valve fixing part 520, when the valve fixing part 520 is rotated toward the main body 510 by operating the handle 523 to close the valve fixing part 520, the handle 112 of the stopcock valve 110 engages with the power transmission coupler 550 on the side of the main body 510, allowing the stopcock valve 110 to be fastened to the valve control device 500. Conversely, when the valve fixing part 520 is rotated in an opening direction by operating the handle 523, the engagement between the handle 112 and the power transmission coupler 550 is released, allowing the stopcock valve 110 to be separated.

That is, in the embodiment of FIG. 7, the valve fixing part 420 is hinge-rotated to connect and disconnect the stopcock valve 110 to and from the power transmission coupler 460 on the main body 410 side, whereas in the embodiment of FIG. 8, the valve fixing part 520 is hinge-rotated to connect and disconnect the stopcock valve 110 to and from the power transmission coupler 550 on the main body 510 side. In both embodiments, the valve position fixation and the connection of the power transmission path are achieved simultaneously by a single operation of hinge rotation. Therefore, the valve can be attached and detached quickly and intuitively.

The worm gear 540 connected to the output shaft of the motor 530 drives the worm wheel gear 541, and the worm wheel gear 541 can transmit a rotational force to the power transmission coupler 550 directly or through gear coupling. The power transmission coupler 550 can contact the handle 112 to rotate the handle 112. That is, the worm wheel gear 541 can transmit the rotational force to the handle 112 either via the power transmission coupler 550 or directly. The worm gear system provides a high reduction ratio (e.g., 20:1 to 80:1) in a single stage. Therefore, a sufficient torque for valve switching can be secured even with a small motor. In addition, when the lead angle of the worm gear 540 is equal to or smaller than a predetermined value, a self-locking characteristic is exhibited, so that reverse rotation caused by an external force can be prevented even when no current is supplied to the motor 530. By doing so, the position of the handle 112 can be maintained even in the event of a power outage or a temporary interruption of the control system.

However, due to the self-locking characteristics, a separate clutch mechanism (e.g., electronic clutch or one-way clutch) may be required to release the engagement of the worm gear 540 during manual override. The installation direction of the motor 530 may be adjusted according to the procedure environment, and the orientation of the handle 523 may also be set to match the user's approach direction.

FIG. 9 illustrates a structure of the power transmission coupler according to one embodiment.

Referring to FIG. 9, the power transmission coupler 550 is a component that transmits a rotational force by contacting the outer surface of the handle 112, and may include a plurality of circular projections 551 formed on the handle contact surface. The circular projections 551 are formed of an elastic material and can transmit a rotational force without slipping through frictional contact with the outer surface of the handle 112.

Since the shape, diameter, and thickness of the handle 112 of the commercially available stopcock valve 110 vary depending on the manufacturer and model, the circular projections 551 may be implemented as a replaceable structure. Specifically, the circular projections 551 can be detachably coupled to the main body of the power transmission coupler 550 by fitting, thread coupling, or magnetic attachment, and may be provided as a kit in multiple sizes corresponding to the sizes of the handle 112.

The material of the circular projections 551 may be at least one of urethane, silicone, thermoplastic elastomer (TPE), or natural rubber. Urethane has excellent wear resistance, silicone has good biocompatibility and chemical resistance, and TPE is suitable for mass production by injection molding. With this replaceable structure, a single actuator 210 can be applied to different stopcock valves 110, and the performance of the device can be maintained by replacing only the circular projections 551 in case of wear or contamination of the circular projections 551.

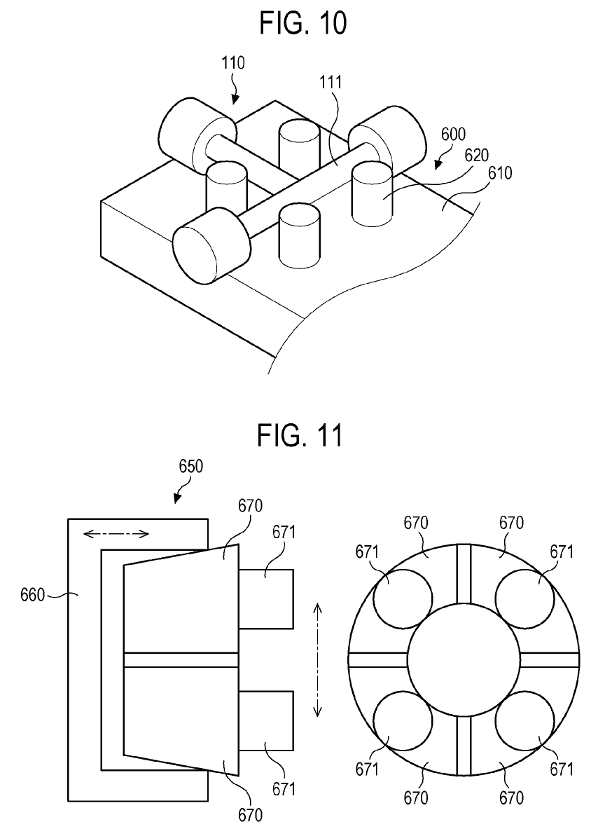

FIG. 10 illustrates a structure of the valve fixing part according to one embodiment.

Referring to FIG. 10, the valve fixing part 600 is an embodiment corresponding to the valve fixing parts 120, 420, and 520 shown in FIGS. 1, 7, and 8, and may be a structure that grips and fixes the outer surface of the lumen housing 111 of the stopcock valve 110. The valve fixing part 600 may include a fixing part body 610 and a plurality of protrusions 620 arranged at predetermined intervals on the fixing part body 610. By inserting the lumen housing 111 into the space formed between the plurality of protrusions 620, the stopcock valve 110 can be easily inserted and withdrawn in the axial direction, while being stably fixed in the rotational and lateral directions.

The protrusions 620 are formed of an elastic material and can provide a fixing force by friction while preventing damage to the lumen housing 111. The elastic material may be at least one of silicone, urethane, TPE, or rubber. The shape of the protrusions 620 may be at least one of a cylindrical shape, a hemispherical shape, a conical shape, or a V-groove shape. A shape that provides the most stable contact at three or more points may be selected according to the shape of the lumen housing 111.

The protrusions 620 may be provided as a kit in multiple sizes corresponding to the sizes of the lumen housing 111. The protrusions 620 that match the stopcock valve 110 to be used before the procedure may be selected and mounted on the fixing part body 610.

FIG. 11 illustrates an automatic chuck type valve fixing part according to another embodiment. FIG. 11 illustrates a modification of the valve fixing part 420 shown in FIG. 7 and the valve fixing part 520 shown in FIG. 8.

Referring to FIG. 11, the automatic chuck type valve fixing part 650 may include an adjustment member 660 and a plurality of jaws 670 that are radially movably coupled to the adjustment member 660. FIG. 11 shows an example in which four jaws 670 are arranged. However, the present disclosure is not limited thereto. A jaw protrusion 671 protruding radially inward may be formed on the inner surface of each jaw 670.

When the adjustment member 660 is rotated, the plurality of jaws 670 can move synchronously in the radial direction by means of an internal interlocking mechanism. That is, when the jaws 670 are moved and retracted synchronously inward in the radial direction by operating the adjustment member 660, the gap between the jaw protrusions 671 of adjacent jaws 670 becomes narrower, and when the jaws 670 are moved and spread apart synchronously outward in the radial direction, the gap between the jaw protrusions 671 becomes wider. After the lumen housing 111 of the stopcock valve 110 is arranged between the jaws 670, when the adjustment member 660 is rotated in a tightening direction, the jaw protrusions 671 come into close contact with the outer surface of the lumen housing 111, allowing the lumen housing 111 to be stably gripped and fixed in the rotational and lateral directions. The interlocking structure of the adjustment member 660 and the jaws 670 shown in FIG. 11 is exemplary, and various mechanisms capable of controlling the radial movement of the jaws 670, such as a scroll mechanism, a spiral cam, or a rack-and-pinion mechanism, may be applied.

Compared to the kit replacement system using the protrusions 620 shown in FIG. 10, the automatic chuck system can accommodate lumen housings 111 having various outer diameters by continuously adjusting the spreading width of the jaws 670 without a kit for each size, and the gripping force is distributed in the circumferential direction, so that the local stress on the lumen housing 111 can be reduced.

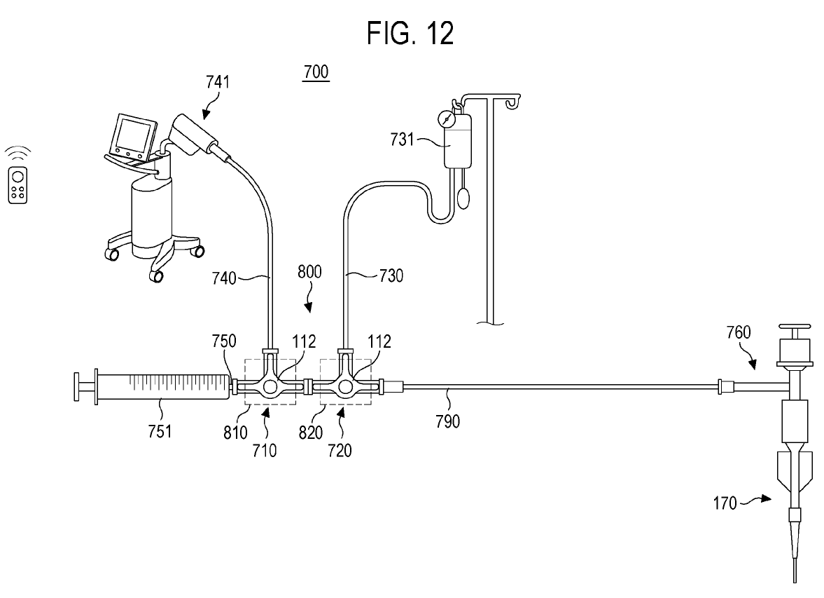

FIG. 12 illustrates a configuration of a fluid management system 700 including a plurality of stopcock valves connected in series according to one embodiment.

Referring to FIG. 12, the fluid management system 700 may include a first three-way stopcock valve (hereinafter, "first stopcock valve") 710, a second three-way stopcock valve (hereinafter, "second stopcock valve") 720 connected in series with the first stopcock valve 710, a saline solution line 730, an automatic contrast agent injector line 740, a manual syringe line 750, an injection line 790, a first valve control device 810 mounted on the first stopcock valve 710 to rotate its handle, and a second valve control device 820 mounted on the second stopcock valve 720 to rotate its handle. Each of the first valve control device 810 and the second valve control device 820 may be implemented as any one of the valve control devices 100, 400, and 500 described with reference to FIGS. 1-11.

Specifically, each of the first stopcock valve 710 and the second stopcock valve 720 is a three-way stopcock valve having a T-shaped flow path and has three ports. In the present disclosure, the ports of each valve are defined as follows based on the flow direction of a fluid flowing toward the first catheter 170 through an injection line 790. That is, in each stopcock valve, the port on a straight flow path located on the side (upstream side) relatively far from the first catheter 170 is referred to as a "second port," the port located on the side (downstream side) relatively close to the first catheter 170 and arranged opposite to the second port on the straight flow path is referred to as a "third port," and the port branched in a direction intersecting the straight flow path between the second port and the third port is referred to as a "first port." According to these criteria, the first stopcock valve 710 is provided with a first port, a second port, and a third port (hereinafter referred to as "the first port of the first stopcock valve 710," "the second port of the first stopcock valve 710," and "the third port of the first stopcock valve 710," respectively). The second stopcock valve 720 is also provided with a first port, a second port, and a third port (hereinafter referred to as "the first port of the second stopcock valve 720," "the second port of the second stopcock valve 720," and "the third port of the second stopcock valve 720," respectively). A manual syringe line 750 is connected to the second port of the first stopcock valve 710, an automatic contrast agent injector line 740 is connected to the first port of the first stopcock valve 710, and the third port of the first stopcock valve 710 is in fluid communication with the second port of the second stopcock valve 720. A saline solution line 730 is connected to the first port of the second stopcock valve 720, and an injection line 790 is connected to the third port of the second stopcock valve 720. That is, due to the series connection from the third port of the first stopcock valve 710 to the second port of the second stopcock valve 720, the fluid flow toward the first catheter 170 is formed using the third port of the second stopcock valve 720 as a final outlet.

With this configuration of series connection, the first stopcock valve 710 performs the function of determining which line, either the automatic contrast agent injector line 740 or the manual syringe line 750, will be selectively connected to the second port of the second stopcock valve 720, and the second stopcock valve 720 performs the function of determining which fluid path, either the fluid path from the first stopcock valve 710 or the fluid path from the saline solution line 730, will be selectively connected to the injection line 790. The expression "control the first stopcock valve 710 through the first valve control device 810" below encompasses the operation in which the controller 260 transmits a control command regarding the rotation direction and rotation angle to the first valve control device 810 to thereby cause the actuator 210 of the first valve control device 810 to rotate the handle of the first stopcock valve 710 at a corresponding angle. The same applies to the second valve control device 820 and the second stopcock valve 720.

The saline solution line 730 extending from a heparin-added saline solution bag 731 can perform the function of continuously cleaning the inside of surgical instruments such as the first catheter 170 and the rotary hemostatic valve while preventing blood coagulation during the procedure. The power injector 741 connected to the automatic contrast agent injector line 740 can inject a contrast agent at a preset flow rate and pressure. The power injector 741 can be remotely controlled to start and stop the injection by a remote button. A syringe 751 connected to the manual syringe line 750 can be used by the operator to manually inject a small amount of fluid contrast agent, embolic material, etc., or to suck air from the line. In the present disclosure, the embolic material is a material injected into a blood vessel through the second catheter 770 to selectively occlude the blood vessel and block a target blood flow, and may include, for example, a microsphere, an embolic coil, a liquid embolic agent (e.g., n-butyl cyanoacrylate (n-BCA) or an ethylene vinyl alcohol copolymer embolic agent), polyvinyl alcohol (PVA) particles, gelatin sponge particles, and the like. The embolic material may be appropriately selected depending on the purpose of the procedure and the target blood vessel.

The output side of the fluid management system 700 may include an injection line 790 that is in fluid communication with the first catheter 170 through a side port of a Y-connector 760. In one embodiment, the first valve control device 810 and the second valve control device 820 may be integrated within a single module housing so that the sequence control described below can be performed by a single controller 260. In another embodiment, each valve control device may be housed in an individual housing and installed independently.

In the fluid management system 700 of the present disclosure, the number of stopcock valves is not limited to two. The configuration of FIG. 12 is intended to illustrate an example in which two stopcock valves 710 and 720 are connected in series. In other embodiments, N three-way stopcock valves (where N is an integer of 3 or more) may be connected in series or connected in a topology that combines series connection and parallel connection. Each stopcock valve may be equipped with a corresponding valve control device, namely a first valve control device 810, a second valve control device 820, and an additional N-th valve control device. In such an N-valve configuration, various fluid lines, such as a plurality of contrast agent lines, a plurality of embolic material lines, a cleaning fluid line, a guide wire perfusion line, and the like may be connected to the ports of the respective stopcock valves according to the type and number of fluid lines required in the procedure. The controller 260 can automatically switch to a wider variety of fluid path combinations by controlling the N valve control devices individually or synchronously. In the following embodiments, for the sake of convenience of description, the configuration including two stopcock valves 710 and 720 will be described mainly, but the principle of the corresponding valve control sequence can be extended and applied in the same way to the configurations of N valves.

In addition, when specific angles such as "45-degree rotation" or "90-degree rotation" are mentioned regarding valve rotation in the following embodiments, these angles are exemplary representative values and are not limited to strict numerical values. That is, although the handle of a three-way stopcock valve is designed so that a specific flow path connection state can be achieved at a precise angle due to its structure, in reality, there may be a certain degree of angle deviation due to machining tolerances of the valve handle, assembly tolerances of the valve control device, and control errors of the sensor and actuator. The "45-degree" or "90-degree" rotation described in each embodiment of the present disclosure should be understood as encompassing a rotation within a predetermined allowable tolerance, for example, ±5 degrees, from the angle as a nominal value, or within an angle range where the flow path communication state is stably maintained. Furthermore, if the amount of rotation required varies depending on the internal port shape and flow path angle of the valve, the "45-degree" or "90-degree" rotation should be understood to mean a rotation by the corresponding angle. In other words, as long as the fluid communication state achieved by the rotation of a nominal angle value is maintained, an angular deviation within a range capable of forming a substantially identical fluid communication state falls within the scope of the present disclosure.

The fluid path control method according to one embodiment of the present disclosure is a method in which, in the above-described fluid management system 700, the controller 260 controls the first valve control device 810 and the second valve control device 820 to automatically switch the respective handles of the first stopcock valve 710 and the second stopcock valve 720 in a predetermined order, thereby sequentially switching the fluid path communicating with the injection line 790 in correspondence with each stage of the procedure. That is, in the fluid path control method of the present embodiment, the rotation of the first stopcock valve 710 is performed by the first valve control device 810, and the rotation of the second stopcock valve 720 is performed by the second valve control device 820. Such rotation operations are performed individually or synchronously by control commands transmitted from the controller 260 to the respective valve control devices. In the present disclosure, the controller 260 may include one or more controllers. Different valve control devices may be controlled by different controllers. The controller 260 controls the physical rotation angle of the valve in conformity with each step, thereby forming a state in which the corresponding fluid can move along a specific path according to the operation of a syringe or an injector.

The fluid path control method of the present disclosure includes a first control sequence P1 and a second control sequence P2 depending on the type of procedure, and these control sequences may be selectively executed as follows depending on the type of procedure. In the case of angiography, the first control sequence P1, which injects a contrast agent into a blood vessel through the first catheter 170, may be performed repeatedly. In the case of embolization or thrombectomy, the first control sequence P1 may be performed first, and then the second control sequence P2, which injects a contrast agent or an embolic material into a blood vessel through the second catheter 770, may be performed subsequently.

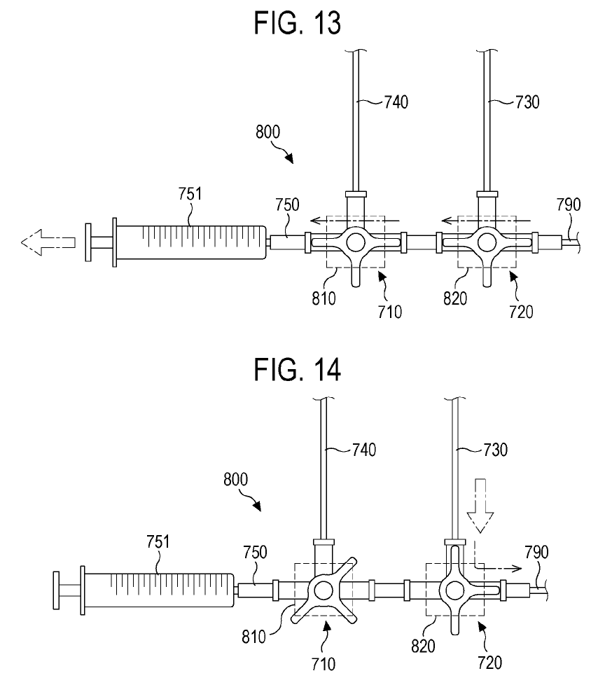

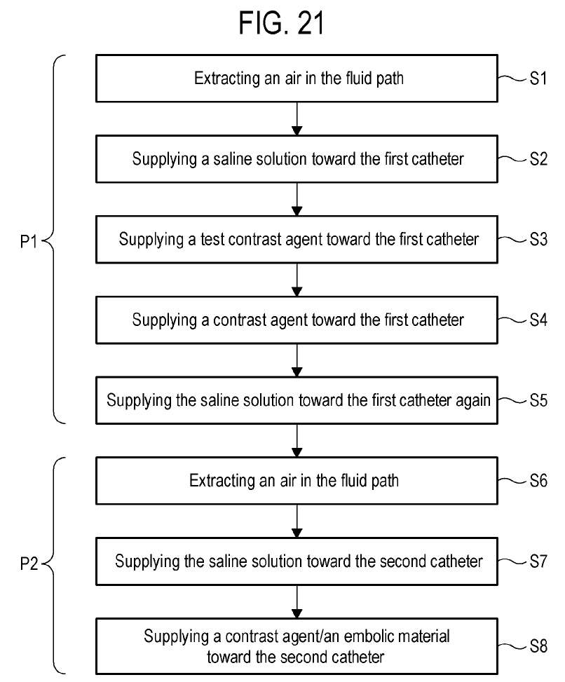

Each of the following steps corresponds to a corresponding step in the overall flow illustrated in a flowchart of FIG. 21. The first control sequence P1 is a set of a series of valve rotation operations constituting a single cycle of angiography, and may include the following first step S1 to fifth step S5. Each valve rotation constituting the first control sequence P1 is applied to the handle of the first stopcock valve 710 or the second stopcock valve 720 via the actuator 210 of each valve control device by the controller 260 transmitting control commands regarding the rotation direction and rotation angle to the first valve control device 810 and/or the second valve control device 820. The specific rotation directions and rotation angles of the first stopcock valve 710 and the second stopcock valve 720 at each step are illustrated in FIGS. 13-17, respectively.

First, as a first step S1, referring to FIG. 13, the controller 260 rotates the first stopcock valve 710 through the first valve control device 810 into a posture in which the manual syringe line 750 communicates with the second port of the second stopcock valve 720, and rotates the second stopcock valve 720 through the second valve control device 820 into a posture in which the second port communicates with the injection line 790. By doing so, a straight fluid path is formed from the syringe 751 to the injection line 790 through the first stopcock valve 710 and the second stopcock valve 720. By retracting the plunger of the syringe 751 in this state, the air remaining in the line is sucked toward the syringe 751 side (see the left arrow of the syringe 751 in FIG. 13). This air extraction may be performed manually by the operator directly manipulating the syringe 751, or by an automatic suction device coupled to the syringe 751. The air extraction may be omitted if the air in the line has already been removed. While step S1 is being performed, both the saline solution line 730 and the automatic contrast agent injector line 740 are kept blocked.

When the air extraction of the first step S1 is completed, the connection of the saline solution line is performed as the second step S2, as shown in FIG. 14. Specifically, the controller 260 rotates the first stopcock valve 710 counterclockwise by 45 degrees through the first valve control device 810. By doing so, the communication between the manual syringe line 750 and the automatic contrast agent injector line 740 and the second port of the second stopcock valve 720 is blocked. The controller 260 rotates the second stopcock valve 720 counterclockwise by 90 degrees through the second valve control device 820, thereby bringing the saline solution line 730 into communication with the injection line 790, so that the saline solution is supplied from the heparin-added saline solution bag 731 toward the first catheter 170.

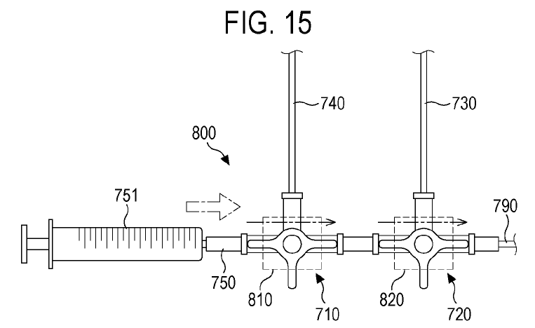

Next, as a third step S3, a test contrast agent is supplied using the syringe 751 to check whether the contrast agent is properly supplied to the intended region through the first catheter 170 located within the blood vessel. That is, referring to FIG. 15, the controller 260 rotates the second stopcock valve 720 clockwise by 90 degrees through the second valve control device 820, thereby blocking communication between the saline solution line 730 and the injection line 790, and bringing the second port of the second stopcock valve 720 into communication with the injection line 790. The controller 260 rotates the first stopcock valve 710 clockwise by 45 degrees via the first valve control device 810, thereby forming a posture in which the manual syringe line 750 communicates with the injection line 790 via the second ports of the first stopcock valve 710 and the second stopcock valve 720. In this state, when the operator advances the plunger of the syringe 751, the contrast agent is supplied to the first catheter 170 (see the right arrow of the syringe 751 in FIG. 15). The operator can determine the position and open state of the first catheter 170 through visual confirmation of this supply.

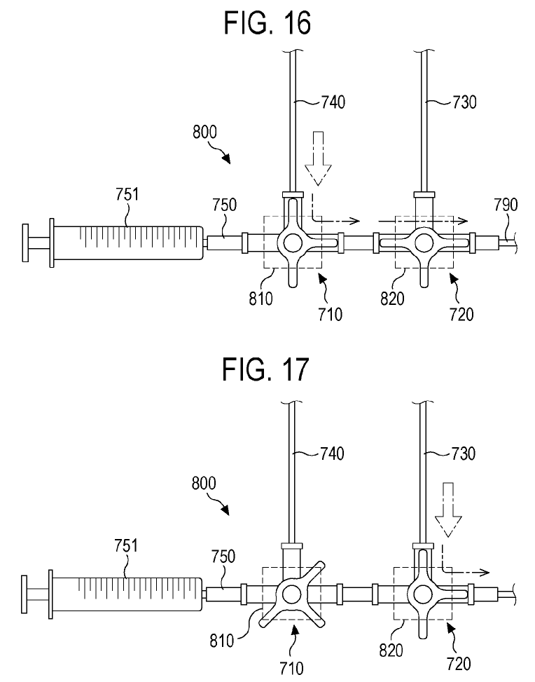

When the position of the first catheter 170 and the open state of the flow path are confirmed by the supply of the contrast agent in the third step S3, a full-scale supply of the contrast agent can be performed by the power injector 741 as a fourth step S4. Specifically, as shown in FIG. 16, the controller 260 controls the second valve control device 820 to maintain the posture of the second stopcock valve 720, while rotating the first stopcock valve 710 counterclockwise by 90 degrees through the first valve control device 810. By doing so, the manual syringe line 750 is blocked and the automatic contrast agent injector line 740 is brought into communication with the second port of the second stopcock valve 720. Thus, the contrast agent is supplied from the power injector 741 toward the first catheter 170 through the injection line 790 at a preset flow rate and pressure. Depending on the operator's choice or the nature of the procedure, the contrast operation may be substituted by supplying the contrast agent in the third step S3 without using the power injector 741, in which case the fourth step S4 may be omitted.

When the supply of the contrast agent is terminated, as a fifth step S5, the saline solution line is reconnected as shown in FIG. 17 to return to the state where the saline solution is supplied to the first catheter 170 again. That is, the controller 260 blocks both the automatic contrast agent injector line 740 and the manual syringe line 750 by rotating the first stopcock valve 710 clockwise by 45 degrees through the first valve control device 810, and brings the saline solution line 730 into communication with the injection line 790 again by rotating the second stopcock valve 720 counterclockwise by 90 degrees through the second valve control device 820. This completes one operation of the first control sequence P1.

During the procedure of angiography, the second step S2 (the supply of the saline solution), the third step S3 and/or the fourth step S4 (the supply of the contrast agent), and the fifth step S5 (the resupply of the saline solution) of the first control sequence P1 may be performed repeatedly as one cycle. This cycle may be repeated any number of times as needed until the end of the procedure. The controller 260 may automatically output parameters such as a rotation direction, a rotation angle, rotation start time and hold time, a contrast agent injection amount, a contrast agent injection pressure, and a cycle interval, which are transmitted to the first valve control device 810 and the second valve control device 820 in each cycle, according to a pre-stored program. The start time of each cycle may be triggered by a direct input from the operator, such as a button input or a voice command, or by an external trigger signal transmitted from the computer 270 to the controller 260. In addition, these parameters may be set by the operator before the procedure begins, or adjusted in real time during the procedure. The controller 260 may save the parameters as a profile for each procedure type and may selectively load and use them when the procedure begins.

Embolization and thrombectomy, which are procedures for injecting drugs into blood vessels or removing blood clots, sequentially use a plurality of procedural tools such as the first catheter 170 corresponding to a guide catheter or diagnostic catheter and the second catheter 770 (e.g., a microcatheter) that advances through the lumen of the first catheter 170. Accordingly, in embolization or thrombectomy, the fluid path control method of the present disclosure may sequentially include a first control sequence P1 performed on the first catheter 170 side and a second control sequence P2 performed subsequently on the second catheter 770 side.

Separate valve sets may be used for the first catheter 170 side and the second catheter 770 side, respectively. In the present disclosure, the valve set arranged on the first catheter 170 side, namely the valve set including the first stopcock valve 710, the second stopcock valve 720, the first valve control device 810, and the second valve control device 820, is referred to as "first valve set 800," and the valve set arranged on the second catheter 770 side, namely the valve set including the third stopcock valve 725 and the third valve control device 825 described below, is referred to as "second valve set 805." The first valve set 800 and the second valve set 805 are unit assemblies that independently switch the fluid path leading to the corresponding first catheter 170 or second catheter 770, respectively, and can be controlled independently or sequentially by the controller 260 within the fluid management system 700.

In this embodiment, the second valve set 805 includes a third stopcock valve 725 arranged on the side of the second catheter 770 and a third valve control device 825 that rotates the handle of the third stopcock valve 725. The third stopcock valve 725 is a three-way stopcock valve. Just like the first stopcock valve 710 and the second stopcock valve 720, the port on the straight flow path located upstream in the flow direction of the fluid flowing toward the second catheter 770 is referred to as the "second port of the third stopcock valve 725," the port located downstream and arranged opposite to the second port on the straight flow path is referred to as the "third port of the third stopcock valve 725," and the port branched in a direction intersecting the straight flow path between the second port and the third port is referred to as the "first port of the third stopcock valve 725." A syringe 751 is fluidly directly connected to the second port of the third stopcock valve 725 to form a manual syringe line 750. A saline solution line (e.g., a line branched from a heparin-added saline solution bag 731 or a line connected to a separate saline solution bag) is connected to the first port of the third stopcock valve 725, and an injection line fluidly connected to the second catheter 770 via a rotary hemostatic valve 780 is connected to the third port of the third stopcock valve 725. The third valve control device 825 may be implemented as any one of the valve control devices 100, 400, and 500 described with reference to FIGS. 1-11, and applies a corresponding rotation amount to the handle of the third stopcock valve 725 in response to a control command from the controller 260.

Meanwhile, in another embodiment, the second valve set 805 may be shared with the second stopcock valve 720 and the second valve control device 820 used on the first catheter 170 side. That is, the second stopcock valve 720 and the second valve control device 820 themselves can be utilized as the second valve set 805 in such a way that after the completion of the first control sequence P1 on the first catheter 170 side, the connection configuration of the fluid management system 700 is changed to connect one port of the second stopcock valve 720 to the rotary hemostatic valve 780 and connect the other port thereof to the syringe 751. In this case, the operations described below for the "third stopcock valve 725" and the "third valve control device 825" can be performed identically by the second stopcock valve 720 and the second valve control device 820. In short, the third stopcock valve 725 and the third valve control device 825 may be a valve and a control device provided separately from the first catheter 170 side, or may be identical to the second stopcock valve 720 and the second valve control device 820 used on the first catheter 170 side.

The second control sequence P2 may include the following sixth step S6 to eighth step S8. In each step, the rotation of the third stopcock valve 725 is applied to the handle of the third stopcock valve 725 via the actuator 210 of the third valve control device 825 by the controller 260 transmitting control commands regarding a rotation direction and rotation angle to the third valve control device 825.

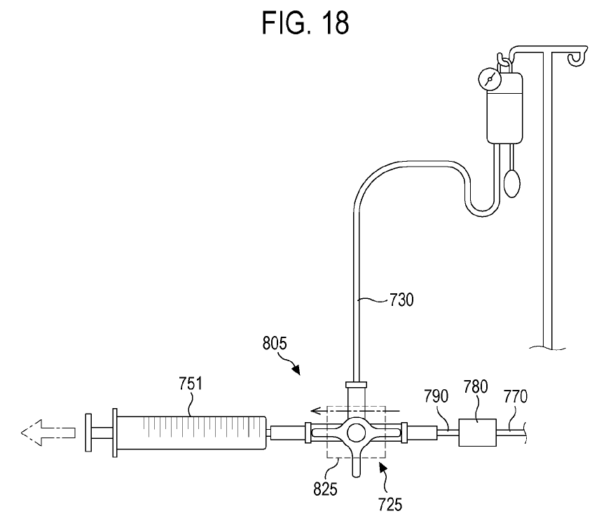

First, as the sixth step S6, referring to FIG. 18, the controller 260 rotates the third stopcock valve 725 in a predetermined direction through the third valve control device 825, thereby forming a posture in which the second port and the third port are in communication with each other while the first port is blocked. By doing so, a straight fluid path is formed from the syringe 751 to the second catheter 770 via the third stopcock valve 725 and the rotary hemostatic valve 780. In this state, by retracting the plunger of the syringe 751, the air within the fluid path is sucked toward the syringe 751 (see the left arrow of the syringe 751 in FIG. 18). The sixth step S6 is preferably performed prior to the actual supply of the contrast agent or embolic agent to the second catheter 770, thereby effectively preventing air embolism into micro-vessels. The sixth step S6 may be repeated during the progression of the second control sequence P2 as needed, or may be omitted if the air in the fluid path on the second catheter 770 side has already been removed.

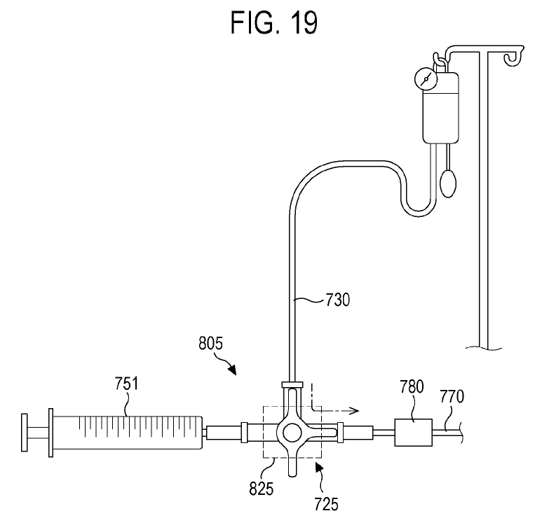

When the air extraction of the sixth step S6 is completed, as a seventh step S7, the supply of a saline solution is performed as shown in FIG. 19. Specifically, the controller 260 rotates the third stopcock valve 725 in a predetermined direction through the third valve control device 825 to block the second port and to bring the first port and the third port into communication with each other. By doing so, the saline solution is continuously supplied into the second catheter 770 via the rotary hemostatic valve 780, thereby flushing the lumen of the second catheter 770 even during the time of changing the procedural tool, and preventing coagulation and thrombus formation during the procedure.

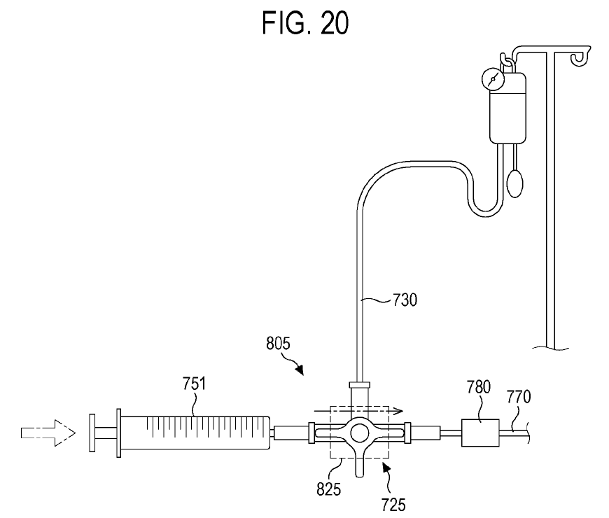

Next, as an eighth step S8, referring to FIG. 20, an operation is performed to precisely supply a contrast agent or an embolic material into micro-vessels through the second catheter 770. Specifically, the controller 260 rotates the third stopcock valve 725 in a predetermined direction through the third valve control device 825 to block the first port and to bring the second port and the third port into communication with each other. In this state, when the operator advances the plunger of the syringe 751, the contrast agent or the embolic material is discharged into the second catheter 770 via the third stopcock valve 725 and the rotary hemostatic valve 780 (see the right arrow of the syringe 751 in FIG. 20). By adopting the manual injection method using the syringe 751 in this way, micro-flow and low-pressure injection, which are difficult to achieve with the power injector 741, can be precisely controlled according to the operator's hand sensitivity, and the oversupply of the embolic material or the damage to micro-vessels can be effectively prevented.

During the course of embolization or thrombectomy, the seventh step S7 of the second control sequence P2 (the connection of the saline solution line) and the eighth step S8 (the injection of the contrast agent or the embolic material) may be performed repeatedly as one cycle, and the sixth step S6 (the air extraction) may be additionally performed at the start of the cycle or between cycles as needed. The repetition of cycles and the execution of each step may all be performed automatically by the collaboration of the controller 260 and the third valve control device 825, and the start of each cycle may be triggered by an input from the operator or a trigger signal from an external system.

FIG. 21 is a flowchart showing an overall flow of the fluid path control method according to one embodiment.

Referring to FIG. 21, the fluid path control method of the present disclosure includes a first control sequence P1 and a second control sequence P2. The first control sequence P1 is performed using the first valve set 800 with respect to the first catheter 170 side and includes first step S1 to fifth step S5. The second control sequence P2 is performed using the second valve set 805 with respect to the second catheter 770 side and includes sixth step S6 to eighth step S8.

In the first control sequence P1, in the first step S1, a fluid path extending from the syringe 751 to the injection line 790 is formed to perform air extraction. In the second step S2, the saline solution line 730 is brought into communication with the injection line 790 to supply a saline solution toward the first catheter 170. In the third step S3, the test supply of the contrast agent is performed by the syringe 751. In the fourth step S4, a full-scale contrast agent supply is performed by the power injector 741. In the fifth step S5, the saline solution line 730 is brought into communication with the injection line 790 again. The second step S2, the third step S3 and/or the fourth step S4, and the fifth step S5 may be repeated as one cycle.

In the second control sequence P2, in the sixth step S6, a fluid path extending from the syringe 751 to the second catheter 770 is formed to perform air extraction. In the seventh step S7, the saline solution is supplied into the second catheter 770. In the eighth step S8, the contrast agent or the embolic material is supplied by the syringe 751. The seventh step S7 and the eighth step S8 may be repeated as one cycle.

In the case of embolization or thrombectomy, the first control sequence P1 may be performed first, followed by the second control sequence P2. In the case of angiography, only the first control sequence P1 may be repeated.

According to the fluid path control method described above, the switching of the handles of the first stopcock valve 710, the second stopcock valve 720, and the third stopcock valve 725 can be performed automatically under the control of the controller 260 by each actuator 210 of the first valve control device 810, the second valve control device 820, and the third valve control device 825. As a result, the repetitive switching operation of the valve handles, which has been conventionally performed manually by an operator or an auxiliary personnel, can be automated. Therefore, the operator is freed from valve operation and can focus on operating the first catheter 170 and the second catheter 770 and reading images. The accuracy and reproducibility of valve switching can be improved, and the risk of infection associated with the manual operation can be reduced.

According to the embodiments of the present disclosure, the valve control device that can be attached to and detached from a commercially available three-way stopcock valve without structural changes is provided, thereby enabling automation of valve switching while maintaining compatibility with existing operating environments.

In addition, the structure of physical separation of the sterile region and the non-sterile region by the sterile barrier allows for automatic valve control by an electromechanical drive system while maintaining a sterile surgical environment, which makes it possible to reduce the risk of infection in the manual operation.

In addition, by the sequence control of the plurality of stopcock valves connected in series, complex fluid path switching required in procedures such as angiography, embolization, and thrombectomy can be performed automatically. Therefore, the operator can be freed from valve operation and can focus on catheter operation and image reading, which makes it possible to improve the accuracy and reproducibility of valve switching.

Although the present disclosure has been described in relation to some embodiments, it should be noted that there may be various modifications and changes without departing from the spirit and scope of the present disclosure, which can be understood by those skilled in the art.

In addition, such modifications and changes should be construed to belong to the scope of the claims appended herein.

What is claimed is:

1. A valve control device for rotating a handle of a three-way stopcock valve, comprising:
an actuator configured to provide a driving force for rotating the handle;
a valve fixing part configured to contact an outer surface of a lumen housing of the stopcock valve to fix the lumen housing; and
a power transmission coupler configured to transmit the driving force of the actuator to the handle.

2. The device of claim 1, which is integrated into a robot system, further comprising:
a controller configured to receive a fluid flow switching command from a robot controller of the robot system and to rotate the handle to a target angle by driving the actuator in response to the fluid flow switching command.

3. The device of claim 2, further comprising:
a sensor configured to detect a rotation state of the handle,
wherein the controller is configured to determine an initial position of the handle based on the rotation state detected by the sensor, calculate an amount of rotation from the initial position to a target position, and input a driving signal corresponding to the amount of rotation to the actuator.

4. The device of claim 3, wherein the power transmission coupler includes a disc coupled to an output shaft of the actuator and at least one driving pin arranged at an eccentric position radially spaced apart by a predetermined distance from a center of rotation of the output shaft on an upper surface of the disc, and configured to apply a driving force by contacting the handle.

5. The device of claim 4, wherein the sensor is a torque sensor configured to detect a load torque when the handle is driven, and
the controller is configured to monitor a driving torque from the torque sensor while rotating the driving pin, and to calculate the initial position of the handle by detecting an angle at which the driving torque exceeds a predetermined threshold value.

6. The device of claim 5, wherein the driving pin is one,
the handle has a T-shaped branch structure, and
the driving pin is arranged on the disc so as to be located within a sector where an angular spacing between two adjacent branches of the handle is 180 degrees.

7. The device of claim 5, wherein the driving pin includes a first driving pin and a second driving pin arranged on the disc at an angular spacing of 90 degrees from each other, and
the controller is configured to calculate the initial position of the handle based on whether a magnitude of the torque detected when the disc is rotated clockwise and counterclockwise is a first torque generated by a single contact between either the first driving pin or the second driving pin and the handle, or a second torque generated by a dual contact between both the first driving pin and the second driving pin and the handle.

8. The device of claim 1, further comprising:
a sterile barrier configured to form a physical boundary between a sterile region and a non-sterile region,
wherein the valve fixing part and the power transmission coupler are arranged in the sterile region, and the actuator is arranged in the non-sterile region.

9. The device of claim 8, wherein the power transmission coupler includes a magnetic coupler,
the magnetic coupler includes a motor-side element including a driving magnet coupled to an output shaft of the actuator and arranged in the non-sterile region, and a driven magnet arranged in the sterile region opposite the driving magnet and configured to transmit a torque to the handle, and
the sterile barrier is interposed between the driving magnet and the driven magnet so that a torque can be transmitted by a magnetic force without physical contact.

10. The device of claim 1, wherein at least one of the valve fixing part, the power transmission coupler, or the actuator is provided by being integrated into a disposable sterile cassette.

11. The device of claim 1, wherein the power transmission coupler includes a first pulley mounted on an output shaft of a motor, a second pulley connected to a rotary shaft of the handle, and a belt stretched between the first pulley and the second pulley to transmit rotational motion.

12. The device of claim 1, wherein the power transmission coupler includes a worm gear connected to an output shaft of a motor, and a worm wheel gear driven by meshing with the worm gear and configured to transmit a rotational force to the handle.

13. The device of claim 1, wherein the valve fixing part is coupled to a main body rotatably around a hinge shaft,
a plurality of fixing pins are formed on an inner surface of the valve fixing part to contact the outer surface of the lumen housing and to restrain the lumen housing in rotational and lateral directions, and
when the valve fixing part is closed by being rotated toward the main body, the lumen housing is fitted and fixed between the plurality of fixing pins, and the handle engages with the power transmission coupler.

14. A valve control device including a plurality of valve control devices for rotating handles of a plurality of three-way stopcock valves, respectively, each of the plurality of valve control devices comprising:
an actuator configured to provide a driving force for rotating a corresponding handle;
a valve fixing part configured to contact an outer surface of a lumen housing of a corresponding stopcock valve to fix the lumen housing; and
a power transmission coupler configured to transmit the driving force of the actuator to the corresponding handle.

15. The device of claim 14, wherein the plurality of three-way stopcock valves include a first stopcock valve and a second stopcock valve connected in series with the first stopcock valve,
the plurality of valve control devices include a first valve control device configured to rotate a handle of the first stopcock valve, and a second valve control device configured to rotate a handle of the second stopcock valve,
each of the first stopcock valve and the second stopcock valve includes a second port on a straight flow path located upstream in a flow direction of a fluid flowing toward a first catheter, a third port located downstream in the flow direction and arranged opposite the second port on the straight flow path, and a first port branched in a direction intersecting the straight flow path between the second port and the third port,
a manual syringe line with a syringe connected thereto is connected to the second port of the first stopcock valve,
an automatic contrast agent injector line with a power injector connected thereto is connected to the first port of the first stopcock valve,
the third port of the first stopcock valve is fluidly connected to the second port of the second stopcock valve,
a saline solution line is connected to the first port of the second stopcock valve,
an injection line fluidly connected to the first catheter is connected to the third port of the second stopcock valve, and
the valve control device further includes a controller configured to perform a first control sequence that sequentially switches a fluid path communicating with the injection line by transmitting a control command to the first valve control device and the second valve control device and rotating each handle of the first stopcock valve and the second stopcock valve.

16. The device of claim 15, wherein the first control sequence includes a first step of rotating the first stopcock valve into a posture in which the manual syringe line communicates with the second port of the second stopcock valve, and rotating the second stopcock valve into a posture in which the second port communicates with the injection line, to thereby form a fluid path extending from the syringe to the injection line so that air within the fluid path can be extracted.

17. The device of claim 16, wherein the first control sequence further includes a second step of, after the first step, rotating the first stopcock valve to block communication between the manual syringe line and the automatic contrast agent injector line and the second port of the second stopcock valve, and rotating the second stopcock valve to bring the saline solution line into communication with the injection line, so that a saline solution can be supplied from the saline solution line toward the first catheter.

18. The device of claim 17, wherein the first control sequence further includes a third step of, after the second step, rotating the second stopcock valve to block communication between the saline solution line and the injection line and to bring the second port of the second stopcock valve into communication with the injection line, and rotating the first stopcock valve to form a posture in which the manual syringe line communicates with the injection line via the second ports of the first stopcock valve and the second stopcock valve, so that a contrast agent can be supplied from the syringe toward the first catheter.

19. The device of claim 18, wherein the first control sequence further includes a fourth step of, after the third step, rotating the first stopcock valve while maintaining the posture of the second stopcock valve to block the manual syringe line and to bring the automatic contrast agent injector line into communication with the second port of the second stopcock valve, so that the contrast agent can be injected from the power injector toward the first catheter through the injection line.

20. The device of claim 19, wherein the first control sequence further includes a fifth step of, after the fourth step, rotating the first stopcock valve to block both the automatic contrast agent injector line and the manual syringe line, and rotating the second stopcock valve to allow the saline solution line to communicate with the injection line again.

21. The device of claim 20, wherein the first control sequence repeatedly performs a cycle including the second step, the third step or the fourth step, and the fifth step.

22. The device of claim 21, wherein the plurality of valve control devices further includes a third valve control device configured to rotate a handle of a third stopcock valve arranged on the side of a second catheter,
the third stopcock valve includes a second port on a straight flow path located upstream in a flow direction of a fluid flowing toward the second catheter, a third port located downstream in the flow direction of the fluid flowing toward the second catheter and arranged opposite the second port of the third stopcock valve on the straight flow path, and a first port branched in a direction intersecting the straight flow path between the second port and the third port of the third stopcock valve,
a manual syringe line with a syringe connected thereto is connected to the second port of the third stopcock valve,
a saline solution line is connected to the first port of the third stopcock valve,
an injection line fluidly connected to the second catheter via a rotary hemostatic valve is connected to the third port of the third stopcock valve, and
the controller is configured to, after the first control sequence, further perform a second control sequence that sequentially switches a fluid path communicating with the second catheter by transmitting a control command to the third valve control device and rotating the handle of the third stopcock valve.

23. The device of claim 22, wherein the second control sequence includes a sixth step of rotating the third stopcock valve to form a posture in which the second port and the third port are in communication with each other while blocking the first port, so that air can be extracted from a fluid path extending from the syringe to the second catheter through the rotary hemostatic valve.

24. The device of claim 23, wherein the second control sequence further includes a seventh step of, after the sixth step, rotating the third stopcock valve to block the second port and to bring the first port and the third port into communication with each other, so that a saline solution can be supplied from the saline solution line into the second catheter via the rotary hemostatic valve.

25. The device of claim 24, wherein the second control sequence further includes an eighth step of, after the seventh step, rotating the third stopcock valve to block the first port and to bring the second port and the third port into communication with each other, so that a contrast agent or an embolic material can be injected from the syringe into the second catheter via the rotary hemostatic valve.

26. The device of claim 25, wherein the second control sequence repeatedly performs a cycle including the seventh step and the eighth step.

27. A fluid path control method for switching a fluid path communicating with an injection line connected to a first catheter by rotating each handle of a first three-way stopcock valve and a second three-way stopcock valve connected in series by a valve control device,
wherein each of the first stopcock valve and the second stopcock valve includes a second port on a straight flow path located upstream in a flow direction of a fluid flowing toward the first catheter, a third port located downstream in the flow direction and arranged opposite the second port on the straight flow path, and a first port branched in a direction intersecting the straight flow path between the second port and the third port,
wherein a manual syringe line with a syringe connected thereto is connected to the second port of the first stopcock valve,
wherein an automatic contrast agent injector line with a power injector connected thereto is connected to the first port of the first stopcock valve,
wherein the third port of the first stopcock valve is fluidly connected to the second port of the second stopcock valve,
wherein a saline solution line is connected to the first port of the second stopcock valve, and
wherein the injection line is connected to the third port of the second stopcock valve,
the fluid path control method comprising a first control sequence in which a controller automatically switches the handle of the first stopcock valve and the handle of the second stopcock valve in a predetermined order.

28. The method of claim 27, wherein the first control sequence includes a first step of rotating the first stopcock valve into a posture in which the manual syringe line communicates with the second port of the second stopcock valve, and rotating the second stopcock valve into a posture in which the second port communicates with the injection line, to thereby form a fluid path extending from the syringe to the injection line so that air within the fluid path can be extracted.

29. The method of claim 28, wherein the first control sequence further includes a second step of, after the first step, rotating the first stopcock valve to block communication between the manual syringe line and the automatic contrast agent injector line and the second port of the second stopcock valve, and rotating the second stopcock valve to bring the saline solution line into communication with the injection line, so that a saline solution can be supplied toward the first catheter.

30. The method of claim 29, wherein the first control sequence further includes a third step of, after the second step, rotating the second stopcock valve to block communication between the saline solution line and the injection line and to bring the second port of the second stopcock valve into communication with the injection line, and rotating the first stopcock valve to form a posture in which the manual syringe line communicates with the injection line via the second ports of the first stopcock valve and the second stopcock valve, so that a contrast agent can be supplied from the syringe toward the first catheter.

31. The method of claim 30, wherein the first control sequence further includes a fourth step of, after the third step, rotating the first stopcock valve while maintaining the posture of the second stopcock valve to block the manual syringe line and to bring the automatic contrast agent injector line into communication with the second port of the second stopcock valve, so that the contrast agent can be injected from the power injector toward the first catheter through the injection line.

32. The method of claim 31, wherein the first control sequence further includes a fifth step of, after the fourth step, rotating the first stopcock valve to block both the automatic contrast agent injector line and the manual syringe line, and rotating the second stopcock valve to allow the saline solution line to communicate with the injection line again.

33. The method of claim 32, wherein the first control sequence repeatedly performs a cycle including the second step, the third step or the fourth step, and the fifth step.

34. The method of claim 32, further comprising: a second control sequence that rotates a handle of a third stopcock valve arranged on the side of a second catheter by a third valve control device,
wherein the third stopcock valve includes a second port on a straight flow path located upstream in a flow direction of a fluid flowing toward the second catheter, a third port located downstream in the flow direction of the fluid flowing toward the second catheter and arranged opposite the second port of the third stopcock valve on the straight flow path, and a first port branched in a direction intersecting the straight flow path between the second port and the third port of the third stopcock valve,
a syringe is connected to the second port of the third stopcock valve,
a saline solution line is connected to the first port of the third stopcock valve,
an injection line fluidly connected to the second catheter via a rotary hemostatic valve is connected to the third port of the third stopcock valve, and
the second control sequence is performed after the first control sequence, and includes a sixth step of rotating the third stopcock valve to form a posture in which the second port and the third port are in communication with each other while blocking the first port, so that air can be extracted from a fluid path extending from the syringe to the second catheter through the rotary hemostatic valve.

35. The method of claim 34, wherein the second control sequence further includes a seventh step of, after the sixth step, rotating the third stopcock valve to block the second port and to bring the first port and the third port into communication with each other, so that a saline solution can be supplied from the saline solution line into the second catheter via the rotary hemostatic valve.

36. The method of claim 35, wherein the second control sequence further includes an eighth step of, after the seventh step, rotating the third stopcock valve to block the first port and to bring the second port and the third port into communication with each other, so that a contrast agent or an embolic material can be injected from the syringe into the second catheter via the rotary hemostatic valve.

37. The method of claim 36, wherein the second control sequence repeatedly performs a cycle including the seventh step and the eighth step.
